(19) United States
(12) Patent Application Publication
(10) Pub. No.: US 2018/0056053 A1
(43) Pub. Date: Mar. 1, 2018

(54) PROTRUDING MICROSTRUCTURE FOR TRANSDERMAL DELIVERY

(71) Applicant: JUVIC INC., Seoul (KR)

(72) Inventors: Hyung Il JUNG, Seoul (KR); Mi Roo KIM, Yongin-si (KR); Hui Suk YANG, Seoul (KR); Su Yong KIM, Seoul (KR); Chi Song LEE, Gwangju-si (KR)

(21) Appl. No.: 15/686,845

(22) Filed: Aug. 25, 2017

Related U.S. Application Data
(60) Provisional application No. 62/379,968, filed on Aug. 26, 2016.

(51) Int. Cl. A61M 37/00; B05D 1/18

(52) U.S. Cl. CPC A61M 37/0015; A61M 2037/0023; A61M 2037/0053; B05D 1/18

(57) ABSTRACT

Provided is a structure for transdermal delivery to deliver a material to a body. The structure for transdermal delivery includes a pillar which is a first dermal infiltration part, a microstructure which is connected to an end of one side of the pillar and serves as a second dermal infiltration part. The microstructure includes a body part formed of a biodegradable viscous composition, and a connection part which includes a first contact surface having viscosity generated by a viscous composition to allow the body part to be connected to the pillar and disposed opposite the end of the one side of the pillar.

### CROSS-REFERENCE TO RELATED APPLICATION

This application claims priority to and the benefit of U.S. Provisional Patent Application No. 62/379,968, filed on Aug. 26, 2016, the disclosure of which is incorporated herein by reference in its entirety.

### BACKGROUND

### 1. Field of the Invention

The present invention relates to a structure for transdermal delivery.

### 2. Discussion of Related Art

Conventional biodegradable microneedle patch products require a separate pressure-sensitive adhesive sheet to attach and fix them to skin for a long time. When a pressure-sensitive adhesive sheet is used, a user can feel irritation, and an allergic reaction may be caused. In addition, it is difficult for the pressure-sensitive adhesive sheet to be applied to a joint region with a lot of movement, skin with curves, or skin with hair.

When a patch is applied to skin, to effectively embed microneedles in the skin, the patch is pressed by fingers. In some cases, there is a difference in force of pressing the patch with fingers, and the force may not be evenly dispersed throughout an infiltration area by the fingers. In addition, although all microneedles on the same array are completely infiltrated into skin, complete dissolution of a polymer matrix in the skin takes several minutes to hours depending on the type of a polymer, and during the period, a user feels uncomfortable.

A variety of papers and patent documents are referred throughout the specification, and their citations are indicated. The disclosures of the cited papers and patent documents are incorporated herein by reference in their entireties to more clearly describe the standard of the field of the art to which the present invention belongs and the scope of the present invention.

### SUMMARY OF THE INVENTION

To overcome problems of the conventional art, the present invention is directed to providing a structure for transdermal delivery which is able to deliver a biodegradable microstructure in a short time (e.g., within one minute) into the skin.

The present invention is also directed to providing a CCDP method for manufacturing a structure for transdermal delivery.

Other purposes and advantages of the present invention will become clearer by the detail description, claims and drawings of the present invention as follows.

According to an aspect of the present invention, to deliver a material into a body, a structure for transdermal delivery, comprising a pillar which is a first dermal infiltration part; and a microstructure which is a second dermal infiltration part, binding to an end of one side of the pillar, is provided. The microstructure includes a body part formed of a biodegradable viscous composition; and a connection part which includes a first contact surface having viscosity generated by the viscous composition to allow the body part to be connected to the pillar and opposite the end of the one side of the pillar.

Here, the connection part may further include a second contact surface further providing a binding force between the microstructure and the pillar. The second contact surface may include a part of the outer surface of the pillar extending from the first contact surface, and have viscosity due to the viscous composition.

Here, the body part of the microstructure includes a first body part extending from the first contact surface and a second body part connected with the first body part. Here, the first body part may be formed to have an increased cross-section from the first contact surface to the second body part, and the second body part may be formed to have a decreased cross-section from the first body part to an end of the second body part.

Here, the first body part and the second body part may be formed in a substantially circular shape, and the first body part may be formed to have a continuously increasing diameter from the first contact surface to the second body part.

Here, the second body part may be formed to have a reduced diameter from the first body part to an end of the second body part.

Here, the second body part may be formed to have a continuously decreasing diameter from the first body part to an end of the second body part.

According to another aspect of the present invention, a method for manufacturing a structure for transdermal delivery having a microstructure, which is formed of a viscous composition at an end of a projecting pillar and, when applied to a body, is able to be separated from the pillar within a predetermined separation time, is provided. The method comprises (a) providing a viscous composition containing a biocompatible or biodegradable material to be delivered into a body; (b) providing a pillar with predetermined shape, length and diameter according to a characteristic of a finally-manufactured microstructure; (c) determining a contact area in which the pillar is in contact with the viscous composition in consideration of the separation time for separating the viscous composition from the pillar in the body; (d) placing the viscous composition at an end of the pillar by contacting the end of the pillar with the viscous composition by the determined contact area; (e) drying the viscous composition placed at the end of the pillar; and (f) forming the microstructure in which the viscous composition is attached to the end of the pillar by sequentially repeating the steps (c) to (e).

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a cross-sectional view of a structure for transdermal delivery according to an exemplary embodiment of the present invention;

FIGS. 2A to 2C are detailed cross-sectional views of the structure for transdermal delivery according to the exemplary embodiment of the present invention;

FIGS. 3A and 3B show another example of the structure for transdermal delivery according to the exemplary embodiment of the present invention;

FIGS. 4A to 4D show still another example of the structure for transdermal delivery according to the exemplary embodiment of the present invention;

FIGS. 5A to 5C are diagrams for an operating process of the structure for transdermal delivery according to the exemplary embodiment of the present invention;

FIGS. 6A to 6C are diagrams for another example of the operating process of the structure for transdermal delivery according to the exemplary embodiment of the present invention;

FIG. 7 is a diagram for a contacting-drying process in a method for manufacturing a structure for transdermal delivery according to another exemplary embodiment of the present invention;

FIGS. 8A and 8B are diagrams for another example of the contacting-drying process in the method for manufacturing a structure for transdermal delivery according to the exemplary embodiment of the present invention;

FIG. 9 is a diagram for a dipping-drying process in the method for manufacturing a structure for transdermal delivery according to the exemplary embodiment of the present invention;

FIG. 10 shows a first example illustrating the step of applying an outward force in the method for manufacturing a structure for transdermal delivery according to the exemplary embodiment of the present invention;

FIG. 11 shows a second example illustrating the step of applying an outward force in the method for manufacturing a structure for transdermal delivery according to the exemplary embodiment of the present invention;

FIG. 12 shows a third example illustrating the step of applying an outward force in the method for manufacturing a structure for transdermal delivery according to the exemplary embodiment of the present invention;

FIG. 13 is a diagram for a first experimental example of manufacturing a structure for transdermal delivery according to an exemplary embodiment of the present invention;

FIGS. 14A to 14D are diagrams for the first experimental example of manufacturing the structure for transdermal delivery in various shapes according to the exemplary embodiment of the present invention;

FIGS. 15A and 15B are photographs showing that each structure for transdermal delivery according to the exemplary embodiment of the present invention is applied to the skin of a pig cadevar;

FIG. 16 shows images of the structure for transdermal delivery according to the exemplary embodiment of the present invention, which is applied to skin at different depths;

FIG. 17 are a graph of a skin penetration force according to an infiltration depth of the structure for transdermal delivery according to the exemplary embodiment of the present invention;

FIGS. 18A and 18B are images of structures for transdermal delivery manufactured with different repeating cycles of a dipping process in a method for manufacturing a structure for transdermal delivery according to another exemplary embodiment of the present invention;

FIG. 19 is a graph illustrating a drug content in a microstructure according to the repeating cycles of the dipping process according to another exemplary embodiment of the present invention;

FIGS. 20A to 20C are images of structures for transdermal delivery manufactured by repeating a dispensing-drying process in another exemplary embodiment of the present invention; and

FIGS. 21A to 21C are images of structures for transdermal delivery manufactured by performing a dispensing process after a dipping process in another exemplary embodiment of the present invention.

### DETAILED DESCRIPTION OF EXEMPLARY EMBODIMENTS

Hereinafter, exemplary embodiments of the present invention will be described in detail with reference to the accompanying drawings to be easily implemented to those of ordinary skill in the art to which the present invention belongs. The present invention may be realized in various conformations, and is not limited to the exemplary embodiments described herein. To clearly describe the present invention with reference to the drawings, parts that are not related to the description will be omitted, and the same reference numerals denote the same or similar components throughout the specification.

It should be understood that the terms “include” and “have” used herein designate the presence of characteristics and components described in the specification, and do not previously exclude the possibility of the presence or addition of at least one different characteristic, or a number, step, action, a component, a part or a combination thereof. In addition, when a portion of a layer, film, region or plate is placed “on” another portion, it may be placed “directly on” that portion, and also have a third portion between these portions. On the other hand, when a part of a layer, film, region or plate is placed “under” another part, it also includes that the part is placed “directly under” another part, and a third part is placed between these parts.

Hereinafter, a structure for transdermal delivery according to an exemplary embodiment of the present invention will be described.

The structure for transdermal delivery according to an exemplary embodiment of the present invention may comprise a pillar and a microstructure.

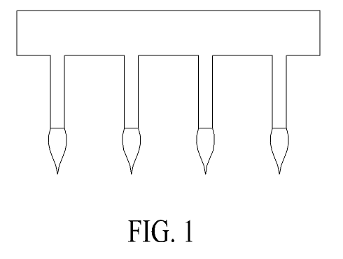

As shown in FIG. 1, the pillar may be formed on a support. The support may be formed as a substrate or patch type. Preferably, the support is formed of a non-pressure-sensitive adhesive material. Selectively, the support may be formed of a pressure-sensitive adhesive material.

As an example, the support may be manufactured of a metal, a polymer (e.g., the above-described biocompatible/biodegradable polymer), an organic chemical, a silicon-based ceramic or a semiconductor material. In addition, the pillar may be integrated with the support.

Generally, the pillar may be formed in a cylindrical shape having a circular cross-section and a uniform diameter, and having a decreased diameter the further away from the support. The cross-section of the pillar may be formed in a polygonal shape.

The pillar may be connected with the support at one side, and may have or be connected with a microstructure at the other side. The pillar supports the microstructure to be spaced a predetermined distance from the support. The length of the pillar is related to the distance of the microstructure from the pillar. This is associated with a body infiltration depth of the microstructure.

As an example, the pillar may have a length of 50 to 10,000 μm and a diameter of 10 to 1000 μm. More specifically, the pillar may have a length of 250 to 3500 μm, and a diameter of 80 to 800 μm.

The pillar may be formed of various materials. The pillar may be formed of a metal, a polymer (e.g., the above-described biocompatible/biodegradable polymer), an organic chemical, a silicon-based ceramic or a semiconductor material.

The pillar may include a plurality of pillar arrays, which are regularly arranged or irregularly arranged on a substrate. The physical parameters of the used pillar may be adjusted.

The adjustable physical parameters of the pillar may include a shape, length or diameter of the pillar. As an example, the pillar may be formed in a conical or cylindrical shape.

As will be described below, the cylindrical pillar may be more rapidly separated from the microstructure, compared to the conical pillar.

In one exemplary embodiment of the present invention, the microstructure may be connected with or formed at the pillar, and may be connected to an end opposite the end of the pillar connected to the support. As will be described below, the microstructure may be connected with or formed at the pillar by a cyclic contact and dry on a pillar (CCDP) method.

Here, when the pillar is in surface contact with the microstructure, they may be connected by a pressure-sensitive adhesive force of the viscous composition forming the microstructure. As will be described below, the surface of the pillar in surface contact with the microstructure may include an end of one side of the pillar, and further include a part of the outer side thereof.

The microstructure may be formed in a substantial candlelight shape.

According to an exemplary embodiment of the present invention, the microstructure formed at the end of one side of the pillar has a shape with curvature or a candlelight shape. The microstructure with curvature makes the pillar easily infiltrated into skin, and can be rapidly separated from the pillar in the infiltrated skin.

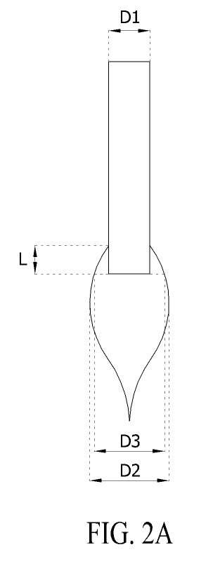

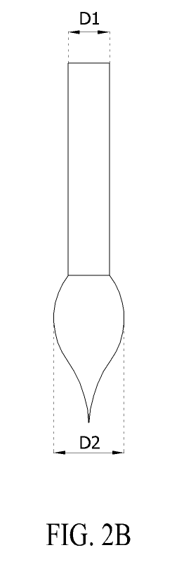

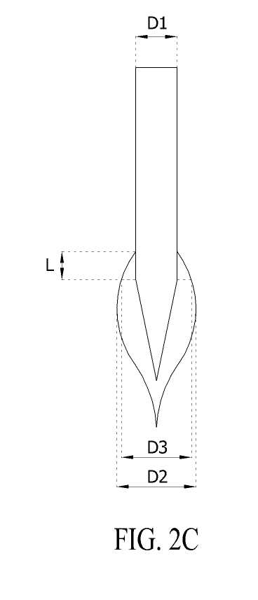

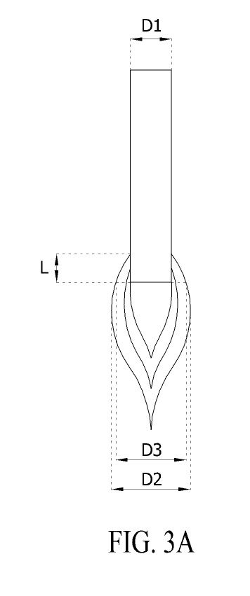

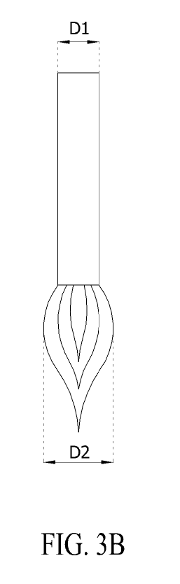

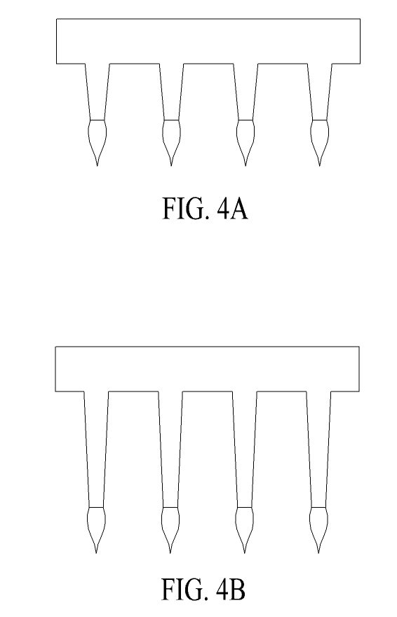

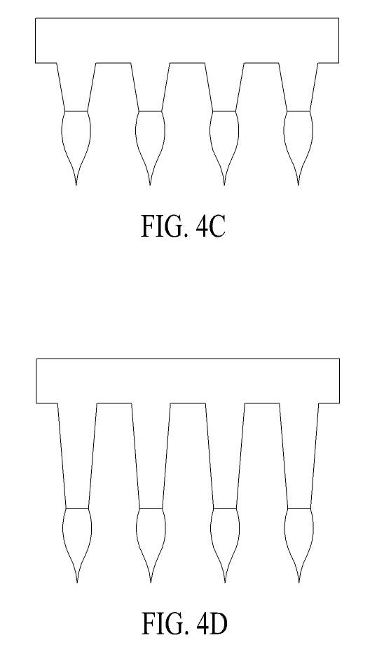

Referring to FIGS. 2 and 3, it can be seen that the pillar is connected with the microstructure in various forms.

A direction in which the microstructure is connected to the pillar is referred to as one side, and the opposite direction is referred to as the other side.

First, in consideration of the cross-section of the microstructure in the lengthwise direction of the pillar, the further away from the pillar, the microstructure has an increased diameter. After the diameter reaches a predetermined value, the diameter is decreased again. Preferably, the end of the microstructure may be formed to be pointed.

That is, in consideration of the cross-section of the microstructure, from one side to the other side, a slope is continuously changed. That is, the outer surface of the microstructure is overall curved and projects outwards.

A rate of increasing the diameter, that is, a slope of the cross-section may be gradually increased to 0, then decreased. Here, a location at which the slope is 0 may be considered the zenith of the diameter of the microstructure, and the location with the maximum diameter.

The microstructure includes a body part and a connection part, in which the connection part includes a first contact surface connected with the end of the pillar. The first contact surface is formed to be opposite to the end of the pillar, and may have viscosity generated by a viscous composition constituting the microstructure.

The body part may extend in the lengthwise direction of the pillar from the first contact surface.

The connection part may further include a second contact surface, which may be connected with the outer surface of the pillar. As will be described below, the connection part may be manufactured by a dipping method. The second contact surface may be connected from the first contact surface, and may be formed on the outer surface of the end of the pillar. The second contact surface may also be formed to have viscosity due to the viscous composition.

Referring to FIGS. 1 to 3, provided that a diameter of the first contact surface is set as D2, and a diameter of the pillar is set as D1, when the microstructure is manufactured by a contact method, D2 and D1 are substantially the same.

In another exemplary embodiment manufactured by a dipping method, provided that the diameter of a virtual plane of the first contact surface extending virtually and crossing the microstructure is set as “D2′,” D2 is larger than D1.

In addition, D3 refers to the maximum diameter of the body part.

The body part may include a first body part with a continuously increasing diameter from the first contact surface and a second body part connected to the first body part and having a decreasing diameter from a connection surface connected with the first body part.

The connection surface is a cross-section connecting the first body part with the second body part, and having the maximum diameter of the body part. Here, provided that a diameter of the connection surface is set as D3, the maximum diameter D3 may be placed in approximately the middle of the microstructure.

In addition, D3 is larger than D2, D1 and D2′.

Accordingly, when the structure for transdermal delivery infiltrated into skin is removed from the skin, the pillar having a relatively small diameter may be easily removed, and the microstructure having a relatively large diameter may remain in the body.

L is a parameter that is able to adjust a binding force between the pillar and the microstructure when the outer surface of the end of the pillar is connected with the microstructure.

L is defined as the depth of dipping. The deeper the depth of dipping, the stronger binding force between the microstructure and the pillar, and the structure for transdermal delivery may be stably stored. Meanwhile, when the structure for transdermal delivery and the microstructure are infiltrated into the skin, the time for separating the microstructure may be longer.

In one exemplary embodiment of the present invention, the microstructure may be formed of a viscous composition. The viscous composition refers to a composition which is changed in conformation due to an applied force and has a capability of forming the microstructure.

In addition, the pillar and the microstructure are connected by viscosity of the viscous composition forming the microstructure, and as an area on which the viscosity acts is adjusted, the binding force may be adjusted.

Hereinafter, various modifications for the microstructure will be described.

First, FIG. 2A shows a microstructure connected to an outer surface of an end of one side of a pillar, which may be located in a part of the microstructure. In this case, the end of the pillar and a part of the outer side of the pillar are in contact with the microstructure.

FIG. 2B shows a microstructure connected only to an end of one side of a pillar, which is connected to the end, and thus is not located in the microstructure. In this case, the microstructure is connected with the pillar only by a contact between the end of the microstructure and the end of the pillar.

FIG. 3 shows a multi-layered microstructure connected to a pillar. The multi-layered microstructure may be easily manufactured by a CCDP method which will be described below. Here, the microstructure may include a plurality of parts formed of a viscous composition therein.

The multi-layered microstructure may include a first part disposed at the innermost side to be in direct contact with the pillar, a second part disposed at the outer side of the first part, and a third part disposed at the outer side of the second part. The first to third parts are also formed in a candlelight shape.

Preferably, the first part may be formed of a viscous composition having a relatively weaker pressure-sensitive adhesive force to the pillar as compared to the second and third parts. Therefore, in dermal infiltration, the microstructure may be easily separated from the pillar. In addition, a material to be delivered into the body may be usually included in the first part or the second part. The third part may be formed of a viscous composition with a sufficient strength to facilitate dermal infiltration.

As an example, the viscous composition includes hyaluronic acid and a salt thereof, polyvinyl pyrrolidone, a cellulose polymer (e.g., hydroxypropyl methyl cellulose, hydroxymethyl cellulose, hydroxyethyl cellulose, hydroxypropyl cellulose, ethyl hydroxyethyl cellulose, an alkyl cellulose and carboxymethyl cellulose), dextran, gelatin, glycerin, polyethyleneglycol, polysorbate, propyleneglycol, povidone, carbomer, gum ghatti, guar gum, glucomannan, glucosamine, dammer resin, rennet casein, locust bean gum, microfibrillated cellulose, psyllium seed gum, xanthan gum, arabino galactan, arabic gum, alginate, gelatin, gellan gum, carrageenan, karaya gum, curdlan, chitosan, chitin, tara gum, tamarind gum, tragacanth gum, furcelleran, pectin or pullulan.

The viscosity of the viscous composition is not particularly limited, and may be, for example, 10 to 200000 cSt or less.

The viscosity of such a viscous composition may be changed in various manners according to the type of material contained in the composition, a concentration, a temperature or the addition of a viscosity modifying agent, and may be adjusted to correspond to the purpose of the structure for transdermal delivery.

In one exemplary embodiment of the present invention, the viscous composition may include a biosynthetic and/or biodegradable material as a main component. The term “biocompatible material” used herein refers to a material which is substantially non-toxic to a body, chemically inert, and has no immunogenicity. The term “biodegradable material” used herein refers to a material which is able to be degraded by a body fluid in a living body or microorganisms.

The biocompatible and/or biodegradable material may be, for example, poly(methylacrylate) PMMA, polyester, polyhydroxyalkanoates (PHAs), poly(α-hydroxy acid), poly(β-hydroxy acid), poly(3-hydroxybutyrate-co-valerate) (PHBV), poly(3-hydroxyproprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyetherester, polyanhydride, poly(glycolide-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalate, polyphosphazenes, PHA-PEG, an ethylene vinyl alcohol copolymer (EVOH), polyurethane, silicone, polyester, polyolefin, a copolymer of polyisobutylene and ethylene alpha-olefin, a styrene-isobutylene-styrene triblock copolymer, an acryl polymer and a copolymer thereof, a vinyl halide polymer and copolymer, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketone, polyvinyl aromatics, polystyrene, polyvinyl ester, polyvinyl acetate, an ethylene-methyl methacrylate copolymer, an acrylonitrile-styrene copolymer, a copolymer of ABS resin and ethylene-vinyl acetate, polyamide, alkyd resins, polyoxymethylene, polyimide, polyether, polyacrylate, polymethacrylate, polyacrylic acid-co-maleic acid, chitosan, dextran, cellulose, heparin, hyaluronic acid, alginate, inulin, starch or glycogen, and preferably, polyester, polyhydroxyalkanoate (PHA), poly(α-hydroxy acid), poly(β-hydroxy acid), poly(3-hydroxybutyrate-co-valerate) (PHBV), poly(3-hydroxyproprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(esteramide), polycaprolactone, polylactide, polyglycolide, poly(lactide-co-glycolide) (PLGA), polydioxanone, polyorthoester, polyetherester, polyanhydride, poly(glycolide-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalate, polyphosphazenes, PHA-PEG, chitosan, dextran, cellulose, heparin, hyaluronic acid, alginate, inulin, starch or glycogen.

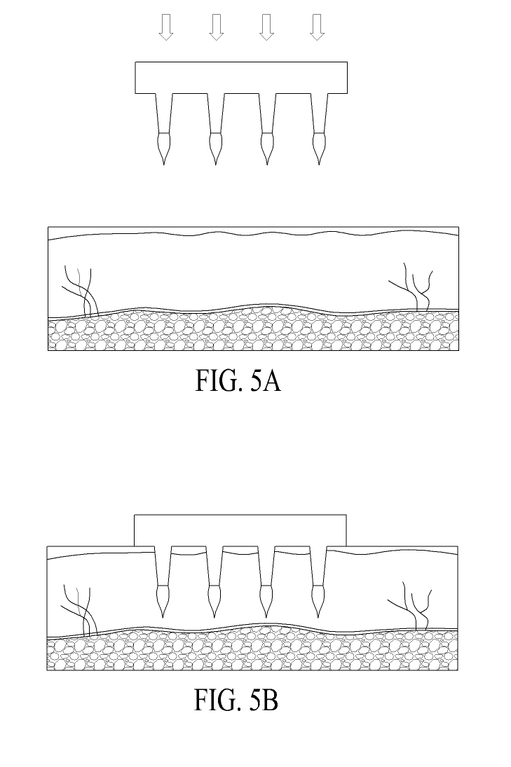

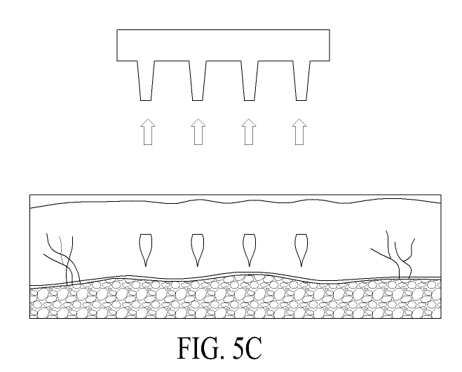

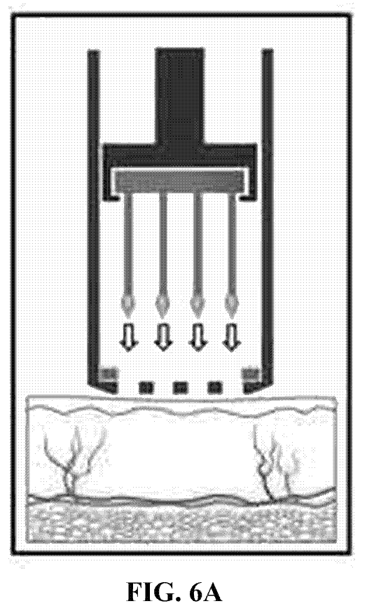

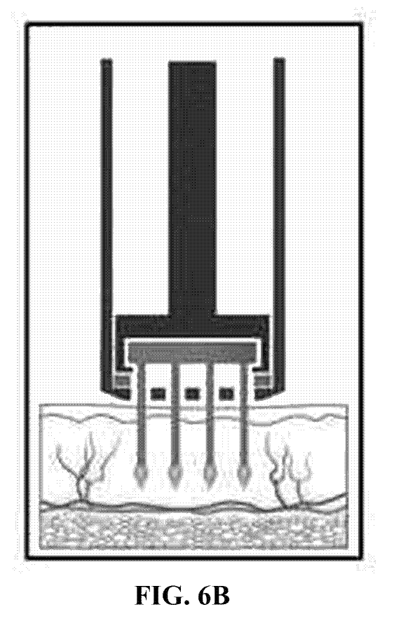

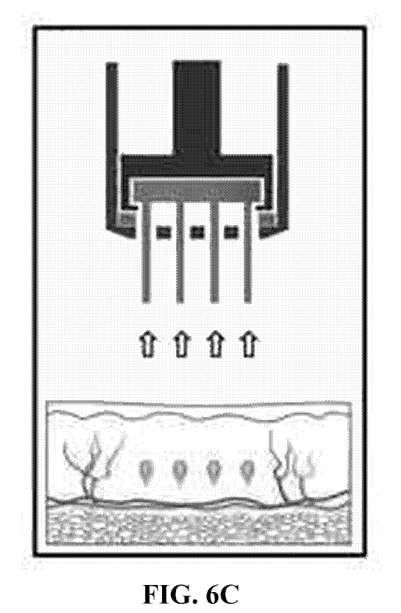

Referring to FIGS. 5 and 6, the operation of the structure for transdermal delivery according to an exemplary embodiment of the present invention will be described.

When the structure for transdermal delivery is inserted into skin, the entire microstructure and a part of the pillar may be infiltrated into the skin. Here, the support is not necessarily a pressure-sensitive adhesive because, unlike the conventional art, it does not need to be attached to the skin for a long time.

Here, according to an infiltration depth of the microstructure, the support may be in contact with the skin or may be spaced a predetermined distance apart. In addition, to increase the maximum infiltration depth, a length of the pillar may be increased. That is, the pillar may adjust the dermal infiltration depth.

After a predetermined time, the support is separated from the skin. Here, the predetermined time may be determined according to a binding force by a contact area between the microstructure and the pillar or a dipping depth.

When the pillar is removed from the skin, the candlelight-like microstructure may remain in the skin. Since the maximum diameter of the microstructure is larger than that of the pillar, it is easy to remove only the pillar.

As an example, the microstructure and the pillar have a separation time of 1 to 200 seconds (e.g., 1 to 60 seconds or 10 to 60 seconds) upon dermal infiltration.

Hereinafter, as another exemplary embodiment of the present invention, a method for manufacturing a structure for transdermal delivery according to an exemplary embodiment of the present invention will be described.

The method for manufacturing a structure for transdermal delivery according to another exemplary embodiment of the present invention may comprise: (a) providing a viscous composition containing a biocompatible or biodegradable material that is to be delivered into a body; (b) providing a pillar having predetermined shape, length and diameter according to a characteristic of a finally-manufactured microstructure; (c) determining a contact area in which the pillar is in contact with the viscous composition in consideration of the separation time for separating the viscous composition from the pillar in the body; (d) placing the viscous composition on an end of the pillar by contacting the end of the pillar with the viscous composition by the determined contact area; (e) drying the viscous composition placed on the end of the pillar; and (f) forming the microstructure in which the viscous composition is attached to the end of the pillar by sequentially repeating the steps (c) to (e).

According to another exemplary embodiment of the present invention, a procedure of manufacturing the structure for transdermal delivery is carried out under a non-heating treatment condition at room temperature or a temperature lower than room temperature (e.g., 5 to 20° C.). Accordingly, in the present invention, a drug that can be used in manufacture of a microstructure includes a drug vulnerable to heat such as a protein drug, a peptide drug, or a nucleic acid molecule for gene therapy.

According to another exemplary embodiment of the present invention, a microstructure including a drug sensitive to heat, such as a protein drug, a peptide drug or a vitamin (preferably, vitamin C), may be manufactured.

(a) Step of Providing Sticky Composition Containing Biocompatible or Biodegradable Material to be Delivered into Body

As described above, the viscous composition used in the present invention contains a biocompatible or biodegradable material. The biocompatible material refers to a material which is substantially not toxic to a body, chemically inert and has no immunogenicity, and the biodegradable material refers to a material which is able to be degraded by a body fluid in a body or microorganisms.

According to another exemplary embodiment of the present invention, the viscous composition further contains a drug or a cosmetic ingredient.

The structure for transdermal delivery manufactured according to another exemplary embodiment of the present invention is used for transdermal administration, and is prepared by mixing a drug with a viscous composition in the preparation of the viscous composition.

In addition, viscosity of the viscous composition may be adjusted by the intrinsic viscosity of the viscous material, and may also be adjusted using an additional viscosity modifying agent as well as the viscous composition.

A viscosity modifying agent conventionally used in the art, for example, hyaluronic acid and a salt thereof, polyvinyl pyrrolidone, a cellulose polymer, dextran, gelatin, glycerin, polyethyleneglycol, polysorbate, propyleneglycol, povidone, carbomer, gum ghatti, guar gum, glucomannan, glucosamine, dammer resin, rennet casein, locust bean gum, microfibrillated cellulose, psyllium seed gum, xanthan gum, arabino galactan, arabic gum, alginate, gelatin, gellan gum, carrageenan, karaya gum, curdlan, chitosan, chitin, tara gum, tamarind gum, tragacanth gum, furcelleran, pectin or pullulan, may be added to a composition containing the main ingredient of the microstructure such as a biocompatible material, thereby suitably adjusting viscosity.

In another exemplary embodiment of the present invention, the mixed drug or cosmetic ingredient is not particularly limited. For example, the drug includes a chemical drug, a protein drug, a peptide drug, a nucleic acid molecule for gene therapy, nanoparticles, an active ingredient of a functional cosmetic, and a cosmetic ingredient.

The drug may include, for example, an anti-inflammatory agent, an analgesic, an anti-arthritic agent, an antispasmodic, an antidepressant, an antipsychotic drug, a tranquilizer, an anxiolytic, a narcotic antagonist, an anti-Parkinson's drug, a cholinergic agonist, an anti-cancer agent, an anti-angiogenesis inhibitor, an immunosuppressant, an antiviral agent, an antibiotic, an appetite suppressant, an anticholinergic drug, an antihistamine, an anti-migraine agent, a hormone agent, a coronary blood vessel, a cerebrovascular or peripheral vasodilator, a contraceptive, an antithrombotic agent, a diuretic, an antihypertensive agent, a drug for a cardiovascular disease, a cosmetic ingredient (e.g., a wrinkle remover, a skin aging inhibitor or skin whitening agent), etc., but the present invention is not limited thereto.

The microstructure of the structure for transdermal delivery according to an exemplary embodiment of the present invention may contain a protein/peptide drug, a hormone, a hormone analogue, an enzyme, an enzyme inhibitor, a signaling protein or a part thereof, an antibody or a part thereof, a single chain antibody, a binding protein or a binding domain thereof, an antigen, an adhesive protein, a structural protein, a regulatory protein, a toxic protein, a cytokine, a transcription regulatory factor, a blood coagulating factor, and a vaccine, but the present invention is not limited thereto.

More specifically, the protein/peptide drug includes insulin, insulin-like growth factor 1 (IGF-1), a growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1 to 13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporine, exedine, lanreotide, luteinizing hormone-releasing hormone (LHRH), nafarelin, parathyroid hormone, pramlintide, enfuvirtide (T-20), thymalfasin and zirconotide.

The viscous composition may also include a material used in transport or delivery of energy such as heat energy, light energy or electrical energy. For example, in photodynamic therapy, to apply light directly to tissue or to a medium such as a light-sensitive molecule, the viscous composition may include a material required to induce light in a specific region in the living body.

The step (a) may include adding a viscosity modifying agent to increase viscosity of the viscous composition, and the viscosity modifying agent that increases the viscosity is the same as described above. Here, the viscosity of the viscous composition may be used to adjust a binding force between the microstructure and the pillar.

(b) Step of Providing Pillar Having Predetermined Shape, Length and Diameter According to Characteristic of Finally Manufactured Microstructure

The pillar may have adjustable physical parameters. Representatively, the parameters include the shape, length or diameter of the pillar.

The length of the pillar is not particularly limited, and is, for example, 1000 to 5000 μm or 2000 to 3500 μm. As an example, the end of the pillar may have a certain cross-section (e.g., 50 to 500 μm²). Here, the binding force with the microstructure may be adjusted according to the cross-section of the pillar.

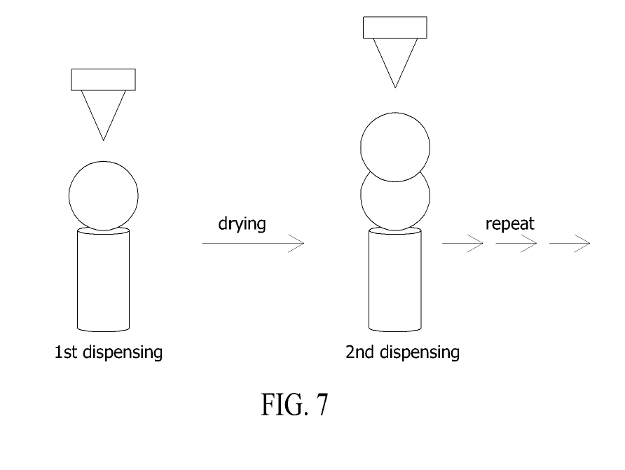

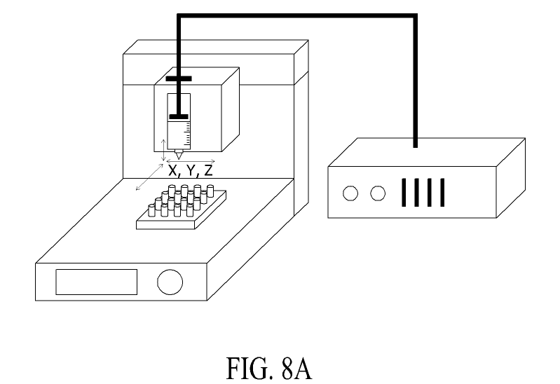

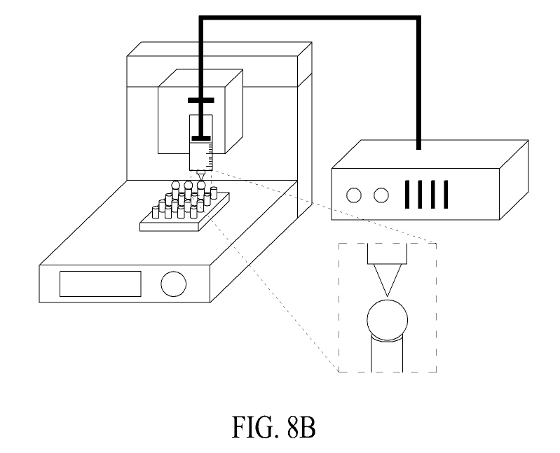

Referring to FIGS. 7 and 8, The length of the pillar may vary according to the dermal infiltration depth of the microstructure. For example, when the dermal infiltration depth is great, the length of the pillar is preferably long. The length of the pillar is highly associated with the maximum infiltrated depth of the microstructure.

In addition, the characteristic of the microstructure formed at the end of the pillar may be adjusted by changing the shape of the pillar. When manufactured with the same dipping depth or the same contact area, a cylindrical pillar, rather than a conical pillar, may be separated from the microstructure in a living body within a shorter time.

(c) Step of Determining Contact Area for Contacting Pillar with Viscous Composition or Dipping Depth in Consideration of Separation Time for Separating Viscous Composition from Pillar in Living Body

(d) Step of Placing Viscous Composition at End of Pillar by Contacting or Dipping End of Pillar to or in a Large Amount of Viscous Composition by the Determined Contact Area or Dipping Depth

The binding force between the microstructure and the pillar may be adjusted according to the contact area between the microstructure and the pillar or dipping depth.

To adjust the binding force, the contact area may be adjusted as follows.

As shown in FIG. 8, the viscous composition may be discharged on a surface of the end of the pillar, thereby forming the microstructure on the pillar. Here, the binding force with the pillar may be adjusted according to the contact area between the viscous composition and the end of the pillar.

When the binding area between the end of the pillar and the viscous composition is large, the binding force is relatively high, and therefore, the viscous composition may be slowly separated from the pillar in a living body. However, the structure for transdermal delivery may be more firmly stored and loaded.

The placement of the viscous composition on the end of the pillar by discharge of the viscous composition may be carried out, for example, using a discharge system. To the discharge system, an outward force (e.g., an air pressure or physical force) used for discharging a viscous solution may be applied. As shown in FIG. 8, the viscous composition is placed at the end of the projected pillar by moving a dispenser along the x, y and z axes.

To adjust the binding force, a dipping depth is adjusted as follows.

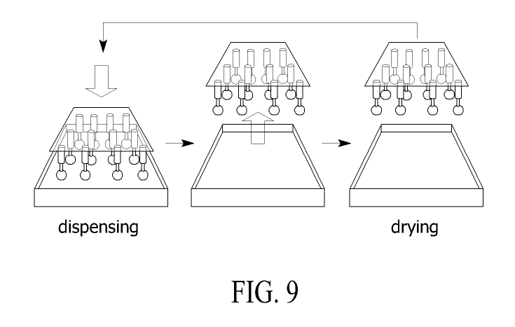

As shown in FIG. 9, the binding force between the pillar and the microstructure may be adjusted by dipping the pillar in the viscous composition. The microstructure may be connected to a part of the outer surface of the pillar. The binding force may be adjusted by the contact area between the pillar and the microstructure, and may be adjusted by a depth of dipping the pillar in the viscous composition.

The time for separating the finally formed microstructure from the pillar (that is, time for separating the microstructure infiltrated into a living body from the pillar) may be adjusted by the dipping depth. The lower the dipping depth, the shorter the time for separating the microstructure infiltrated into the living body from the pillar.

The dipping depth of the pillar may vary, and thus any dipping depth may be applied as long as the viscous composition may be attached to the end of the pillar. For example, the dipping depth may be 100 to 2000 μm, 100 to 800 μm or 200 to 400 μm.

(e) Step of Drying Viscous Composition Placed at End of the Pillar

The drying may be carried out by various methods, for example, by being allowed to stand for drying, drying by air blowing, freeze drying, drying by hot air blowing or drying by natural air blowing. Specifically, the drying in the present invention is carried out by drying by air blowing or drying by natural air blowing.

For example, the drying is carried out by drying the viscous composition of the pillar by natural air blowing for 10 to 60 seconds. By drying, the viscous composition placed (or attached) to the pillar is solidified.

A degree of solidification according to drying may vary depending on the purpose. For example, the solidification includes complete solidification or partial solidification. Drying progresses from the outermost part of the viscous composition, and when surface drying is done at a certain extent, a subsequent process can be performed.

As a modified example, to easily separate the microstructure from the pillar in body infiltration, the first viscous composition may be dried to exhibit a low degree of solidification.

That is, the viscous composition is formed in a first part located in the multi-layered microstructure according to a first drying process in a repeated contacting-drying process. Here, since the first part is directly connected to the pillar, due to overly accomplished solidification, it is impossible to achieve rapid separation in a living body.

(f) Step of Forming Viscous Composition-Attached Microstructure at End of Pillar by Sequentially Repeating the Steps (c) to (e)

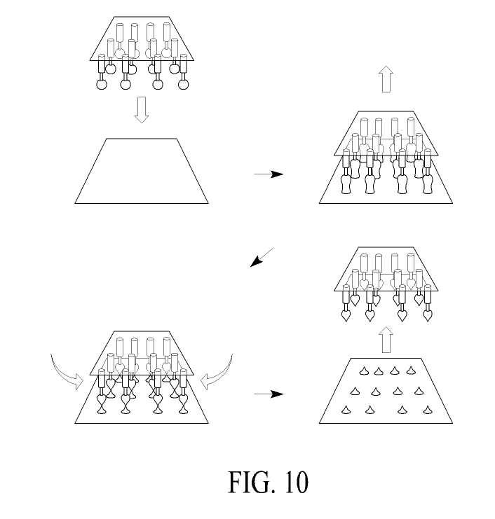

Referring to FIGS. 10, 11 and 12, repetition of contacting and drying allows a sufficient amount of viscous composition to be attached to the end of the pillar. The number of repetition is not limited, and for example, 2 to 100 cycles, 3 to 50 cycles, 3 to 30 cycles, 3 to 20 cycles, 3 to 10 cycles, 2 to 5 cycles, or 2 to 4 cycles.

A structure itself, which is formed at the end of the pillar by the repetition of contacting and drying, may be used as a microstructure.

Selectively, different shapes of microstructures may be obtained by further treating the structure formed at the end of the pillar by the repetition of contacting and drying.

A method for manufacturing a structure for transdermal delivery according to another exemplary embodiment of the present invention may further include applying an outward force to the viscous composition after the step (f).

Following the repetition as described above, before the viscous composition attached to the end of the pillar is solidified (the final drying process is not performed), reducing a diameter of the tip of the microstructure by applying an outward force to the viscous composition placed at the end of the pillar may be further included.

When the diameter of the tip of the microstructure is reduced by applying an outward force to the viscous composition placed at the end of the pillar, the microstructure may be formed as a microneedle.

A method for applying an outward force to the viscous composition placed at the end of the pillar may be performed in various manners.

The first method is described in Korean Patent No. 0793615 invented by the inventors (drawing lithography).

To apply an outward force to the viscous composition, the viscous composition placed at the end of the pillar may be in contact with a plate, and then the pillar may be relatively moved with respect to the plate. (FIG. 9)

For example, following the contacting of the viscous composition placed at the end of the pillar with the plate, the pillar may be moved in a vertical direction with respect to the plate, or after the contacting of the viscous composition placed at the end of the pillar with the plate, the plate may be moved in a vertical direction with respect to the plate.

During relative movement, the viscous composition placed at the end of the pillar extends, and the viscous composition is solidified during extension, thereby finally forming a microstructure. Selectively, the viscous composition extending in the relative movement may be treated by air blowing.

The second method is described in Korean Patent No. 1136738, developed by the inventors (air blowing method). To applying an outward force to a viscous composition, the viscous composition placed at the end of a pillar may be in contact with a plate, and treated by air blowing. Here, the air blowing may be carried out by relatively moving the pillar with respect to the plate.

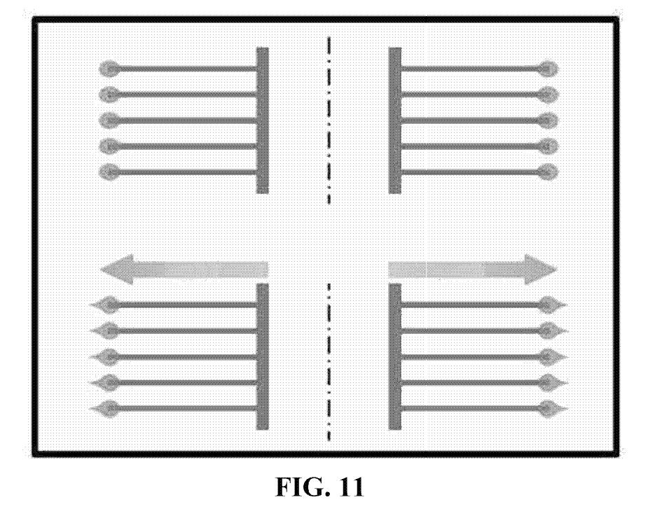

The third method is described in Korean Patent Application No. 2013-0050462, developed by the inventors (centrifugal force method). To apply an outward force to a viscous composition, a centrifugal force may be applied to the viscous composition placed at the end of a pillar. (FIG. 11)

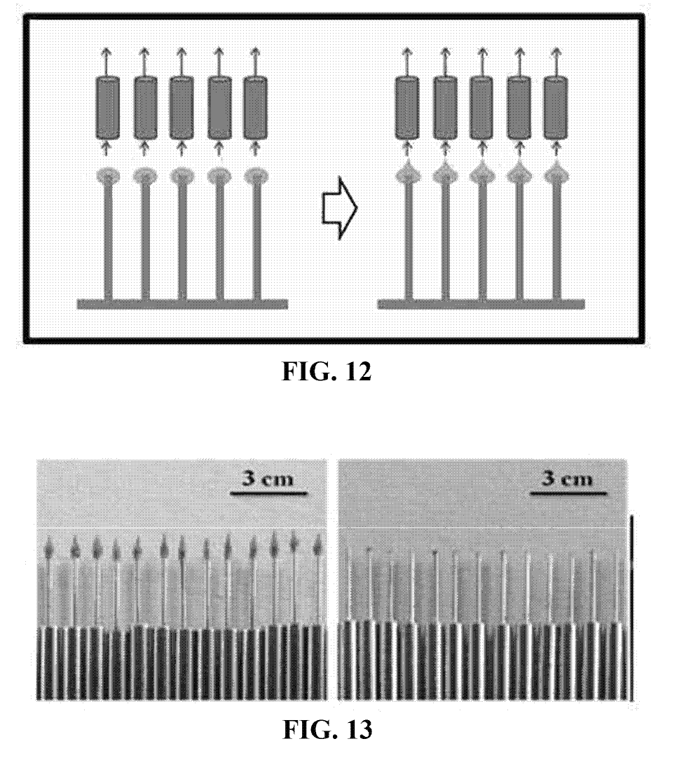

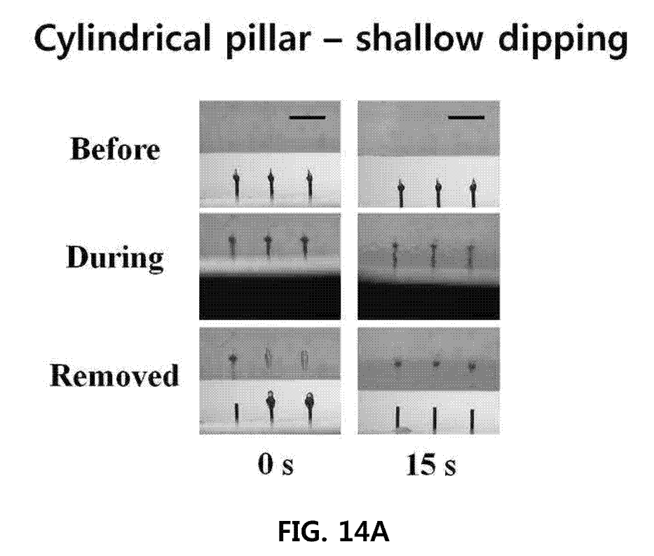

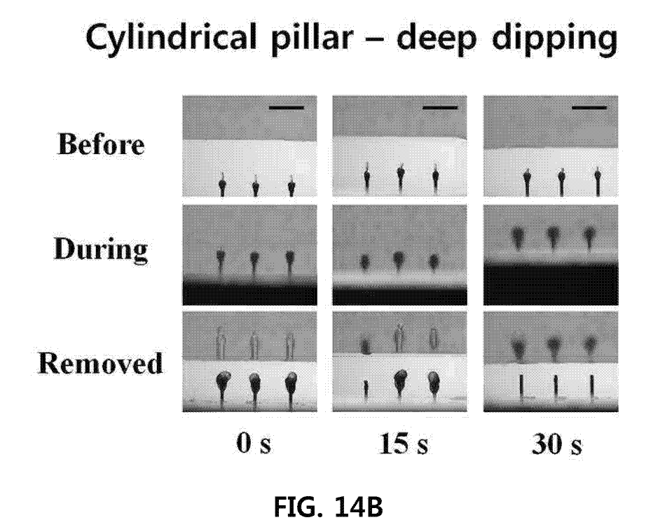

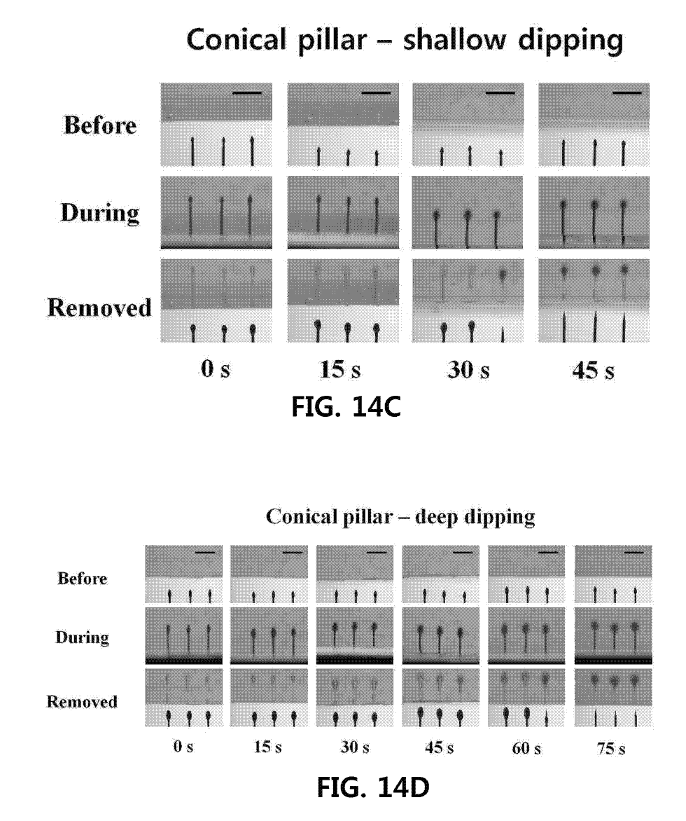

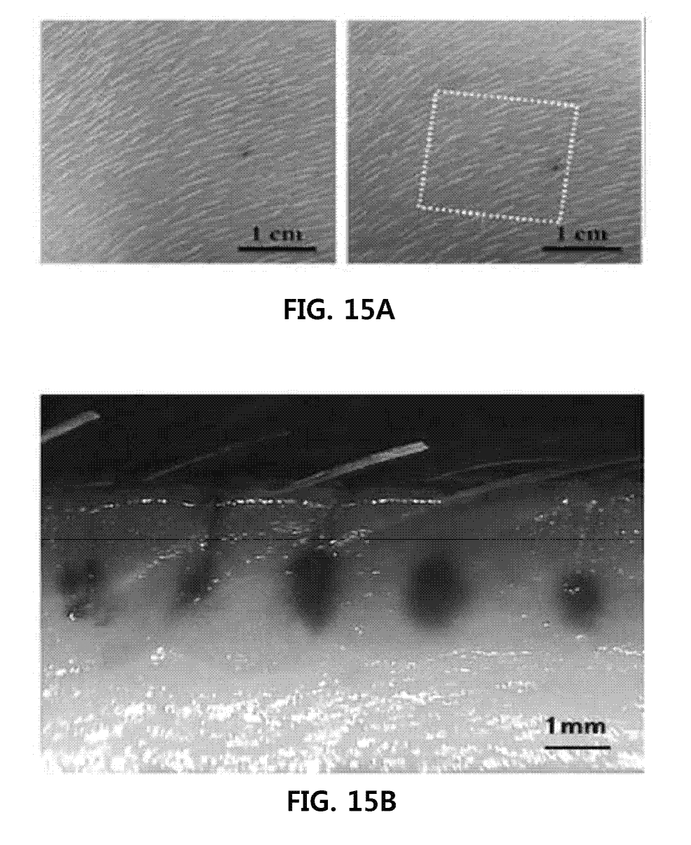

The fourth method is described in Korean Patent Application No. 2013-0019247, developed by the inventors (negative pressure method). To apply an outward force to a viscous composition, negative pressure may be applied to the viscous composition placed at the end of a pillar. (FIG. 12)

The contacting-drying process in the method for manufacturing a structure for transdermal delivery according to another exemplary embodiment of the present invention may be repeated using the same viscous composition, or different viscous compositions.

The step (f) is performed using at least two types of viscous compositions, and inner and outer layers of the microstructure may be formed of different viscous compositions.

According to another exemplary embodiment of the present invention, the inner layer of the microstructure is composed of a viscous composition with a relatively low strength, and the outer layer thereof is composed of a viscous composition with a relatively high strength. As such, a multi-layered microstructure may be manufactured.

For example, when the first and second contacting-drying cycles use “viscous composition A,” and the other contacting-drying cycles use “viscous composition B,” the inner and outer layers of the microstructure may have different characteristics from each other. In addition, the first and second contacting-drying cycles may use a viscous composition without containing a drug, and the other repeating cycles may use a viscous composition with a drug.

Therefore, a drug release pattern in a body may be adjusted using viscous compositions having different release patterns in the contacting-drying process.

As described above, in the microstructure with different inner/outer compositions, although a polymer composition of the first layer placed inside has a weak strength, if the third layer placed in the outermost part has a sufficient strength, the microstructure is able to infiltrate the skin.

On the other hand, when the microstructure is manufactured according to a conventional method using a viscous composition of PVP dissolved in ethanol, a strength effective for dermal infiltration may not be achieved.

However, according to another exemplary embodiment of the present invention, when an inner layer of a microstructure is formed of a PVP viscous composition, and an outer layer thereof is formed of a viscous composition with a sufficient strength (e.g., PVP dissolved in water, carboxymethyl cellulose, hyaluronic acid or chitosan), the microstructure having a strength effective for dermal infiltration may be provided.

Therefore, a microstructure may be manufactured by forming an inner layer thereof using a mixture of hydrophobic drugs only dissolved in an organic solvent such as ethanol, which is not hydrophilic, and a suitable viscous composition (e.g., PVP viscous composition) and forming an outer layer with a different viscous composition.

As another modified example, repetition of contacting and drying may be carried out using a drug-containing viscous composition, and the final contact may be carried out using a drug-free viscous composition, and then an outward force may be applied to a viscous composition placed at the end of the pillar before solidification of the viscous composition attached to the end of the pillar without drying, resulting in the reduction of the diameter of the tip of the microstructure.

In the method for manufacturing a structure for transdermal delivery according to another exemplary embodiment of the present invention, various microstructures, for example, a microneedle, a microblade, a microknife, a microfiber, a microspike, a microprobe, a microbarb, a microarray, or a microelectrode may be used.

A volume of the microstructure of the structure for transdermal delivery finally manufactured through the contacting/drying process is proportional to a content of the polymer contained in the viscous composition. This is because the solvent is evaporated during drying, and only the polymer remains. A polymer material with a large molecular weight has a higher viscosity than the low-molecular weight material although the solution is prepared with the same content.

According to a conventional drawing method, since a microstructure should be manufactured in one process, if a viscosity solution contains an insufficient amount of the polymer, it is impossible to manufacture a microstructure with an effective physical strength.

However, in the method for manufacturing a structure for transdermal delivery according to another exemplary embodiment of the present invention, due to the repetition of a contacting/drying process, a polymer content may be increased, resulting in the manufacture of a structure for transdermal delivery having a microstructure with a sufficient strength.

### Example 1: Adjustment of Time for Separating Microstructure from Pillar (FIG. 13, 14)

Tests were performed to determine the time required for separating biodegradable microneedles from a pillar when applied to skin according to the shape and dipping depth of the pillar. The tests were performed with pillars having conical and cylindrical shapes, and dipping depths of 450 μm and 900 μm.

A viscous composition was prepared by adding a small amount (0.2% w/v) of rhodamine B (Sigma, USA) to a 7% w/v chitosan (Sigma, USA) polymer solution. The number of dipping was three for all samples, and a dipping speed (1.5 mm/min) and a drying time (10 seconds) were equally maintained.

To finally manufacture the microstructure in a microneedle shape, in the final dipping-drying process, the microstructure was rapidly manufactured in a microneedle shape simultaneously with dipping without drying before the viscous composition became dry. The microstructure was manufactured based on drawing lithography at an extension speed of 1.0 mm/min for a drying time of 3 minutes.

For qualitative analysis for the separation of the microneedles from the pillar, a transparent agarose gel (1.4% w/v), rather than real skin, was used. From the time of applying a sample to the agarose gel, separation of the pillar was carried out every 15 seconds, and dyes remaining in the pillar and the gel were examined under a microscope. The experimental result is summarized in Table 1.

| | Micro projection shape | | | |
|---|---|---|---|---|
| | Cylindrical | | Conical | |
| Dipping depth (μm) | 450 | 900 | 450 | 900 |
| Time for separating microneedles | 0-15 | 15-30 | 30-45 | 60-75 |
| from micro projections after | | | | |
| application (sec) | | | | |

As shown in the above result, it was confirmed that, as the dipping depth is smaller, the pillar was more rapidly separated from the microneedles. In addition, it was confirmed that, even with the same dipping depth, the cylindrical pillar is more rapidly separated from the microneedles, compared to the conical pillar. This is because, although dipped to the same depth, the conical type has a smaller contact area that is generated by actual contact with the viscous composition, compared to the cylindrical type. Also, this is because the cylindrical pillar is increased in volume while the viscous composition is caught at the tip of the end thereof during the dipping/drying process, whereas the conical pillar is increased in volume in a lateral direction of the end of the pillar.

### Example 2: Transdermal Delivery of Structure for Transdermal Delivery (FIG. 15)

The structure for transdermal delivery manufactured in Example 1 was applied to skin. The structure for transdermal delivery was applied to unshaved skin (hair length: 2 to 5 mm) of a pig cadaver (infiltration depth: 2-mm depth from dermal surface). Ten seconds after application, the structure for transdermal delivery was separated from the skin.

After the structure for transdermal delivery was infiltrated into the skin for 10 seconds, the microstructure was successfully separated from the pillar. After the structure for transdermal delivery was infiltrated into the skin for 10 seconds, it was confirmed that there was no test drug (rhodamine B) at the end of the pillar and on the dermal surface. In addition, the skin of the applied region was cut in a vertical direction, and then observed under a microscope, thereby confirming that the biodegradable microstructure was completely dissolved in the skin.

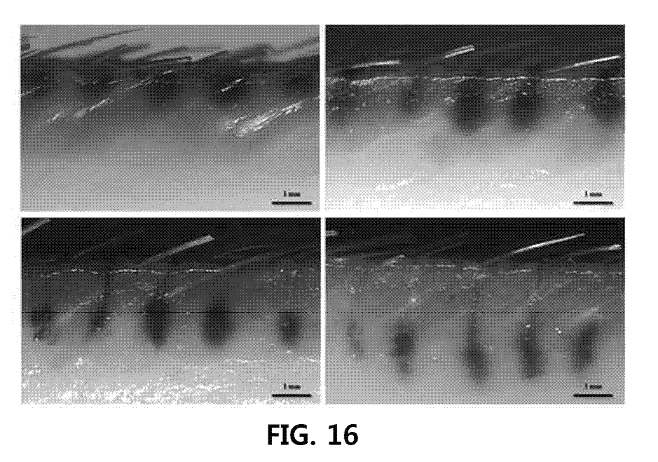

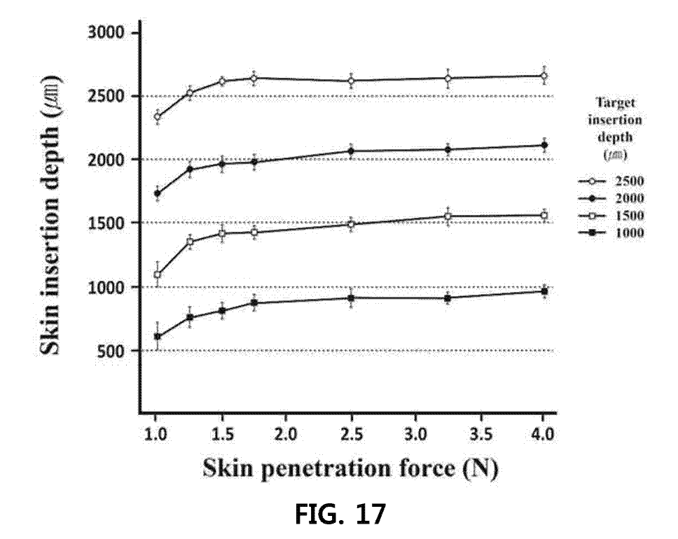

### Example 3: Adjustment of Dermal Infiltration Depth Using Structure for Transdermal Delivery (FIG. 16, 17)

The structure for transdermal delivery manufactured in Example 1 was applied with various dermal infiltration depths (500 to 2000 μm), and after 10 seconds, separated from the skin. Subsequently, the applied region was cut in a vertical direction, and its cross-section was observed under a microscope.

Panels A to D show the results of applying the structure for transdermal delivery to the skin with an infiltration depth of 500 μm, 1000 μm, 1500 μm and 2000 μm. The structure for transdermal delivery of the present invention may be applied to the skin with various dermal infiltration depths.

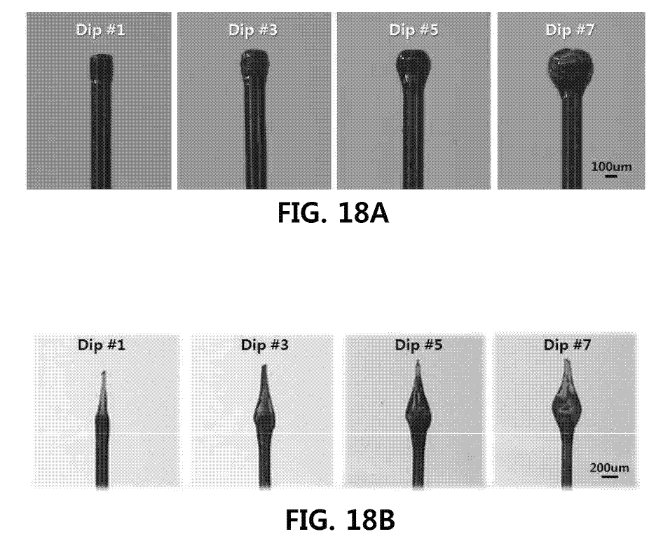

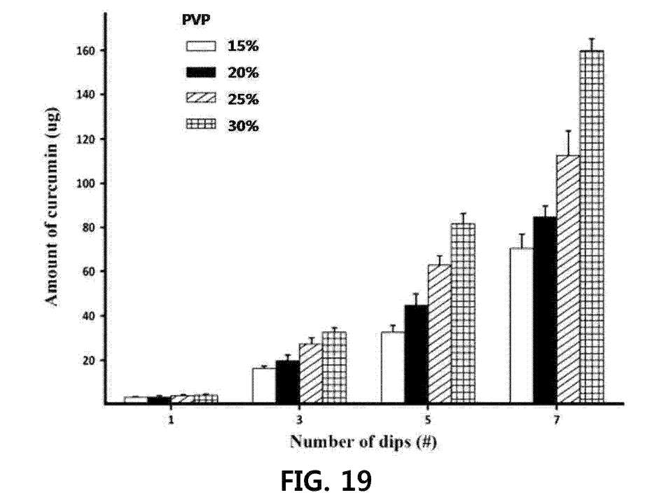

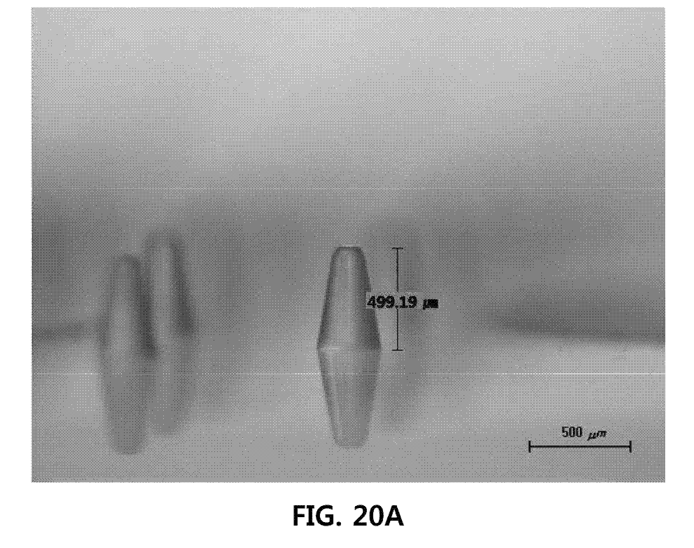

### Example 4: Dipping-Drying (FIG. 18, 19)

The tip of Goryeo Sujichim was processed with a laser to be used as a pillar, and the length of the pillar after laser processing was 2600±35 μm. The pillar was inserted into a fixed frame such that its pointed end faced downward (if needed, the number of pillars is adjustable), and then dipped in the mixed viscous solution of Example 1 (dipping depth: 280±40 μm). Subsequently, the fixed frame was lifted, and then dried with a fan for 15 seconds. The dipping-drying process was repeated once, 3, 5 and 7 times, resulting in the manufacture of a spherical microstructure.

In the final dipping-drying process, the microstructure was rapidly manufactured in a microneedle shape simultaneously with dipping without drying before the viscous composition became dry (FIG. 5b). The microstructure was manufactured based on drawing lithography at an extension speed of 1.0 mm/min for a drying time of 3 minutes.

The pillar with the microstructure was dissolved in a solvent (acetonitrile), and subjected to high performance liquid chromatography (HPLC, Waters 600S, USA) to determine a curcumin content.

When the PVP concentration is the same, it was confirmed that, as the number of repetitions of the dipping/drying process is increased, a volume of the microstructure and a drug content are increased. When the number of repetitions of the dipping-drying process is the same, as the PVP concentration (viscosity) is increased, a larger amount of a drug is loaded. The “Dip # number” represents the number of repetitions of the dipping/drying process.

Accordingly, it can be seen that the amount of a drug loaded in the microstructure can be easily adjusted according to the concentration (viscosity) of a polymer solution and the number of repetitions of the dipping/drying process.

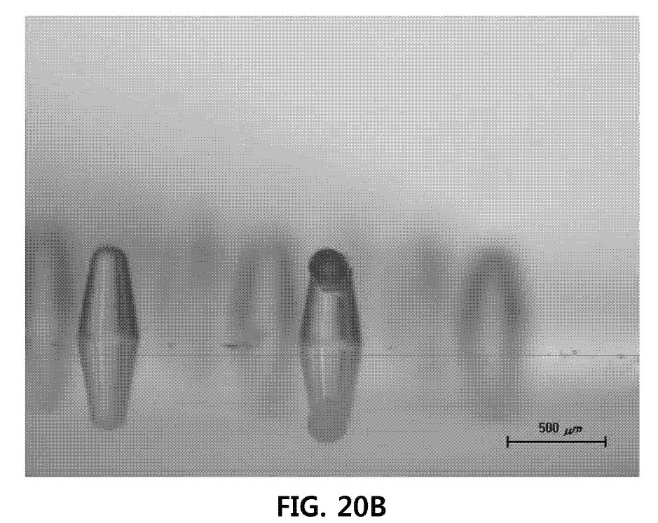

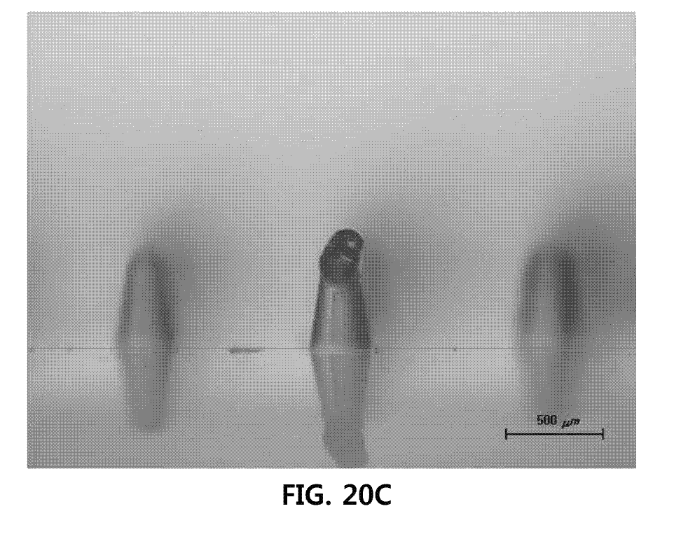

### Example 5: Contacting-Drying-Dispensing (FIG. 20)

A pillar which was formed of poly(methyl methacrylate (PMMA; LG Chem) and had an upper diameter of 125 μm and a length of 500 μm was manufactured by a molding technique (refer to FIG. 7a), and a 10% w/v carboxymethyl cellulose (CMC, low viscosity, Sigma-Aldrich) polymer composition containing Calcein® (fluorescent dye, Sigma-Aldrich) was discharged on the PMMA pillar using a dispenser (Musashi, Japan). After the discharged polymer composition was dried, the same polymer solution was discharged once again in the same manner thereon and dried. A microstructure was formed on the pillar, and the microneedle-like microstructure was finally formed by a suitable extension method (e.g., drawing lithography).

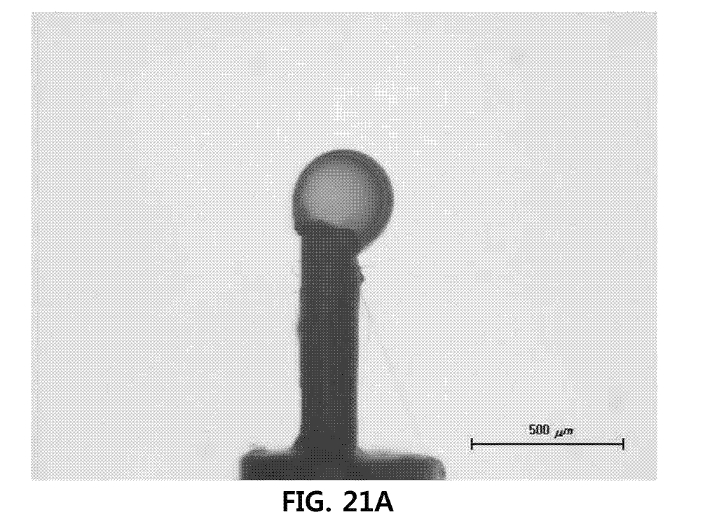

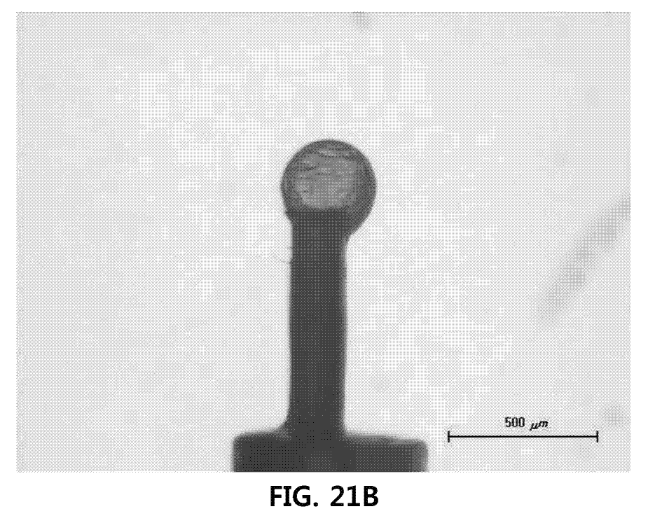

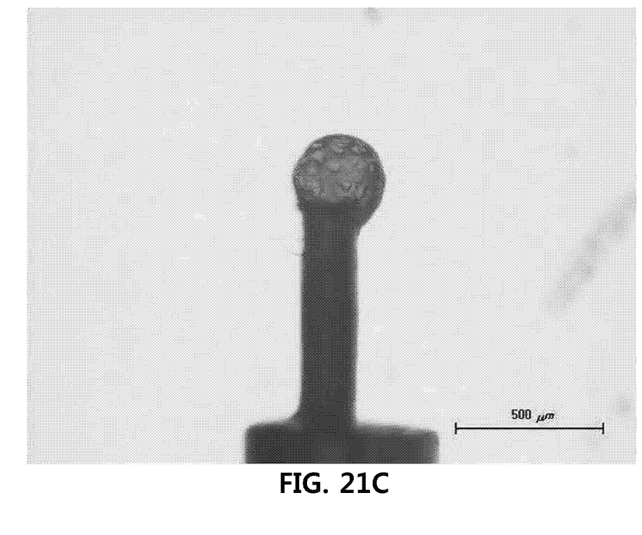

### Example 6: Dipping-Drying-Dispensing (FIG. 21)

An end of a pillar which was formed of SUS and had an upper diameter of 190 μm was dipped once in a 50% w/v PVP (10,000 MW, Sigma-Aldrich) solution so that the polymer composition was attached to the end. A 50% w/v PVP polymer composition containing curcumin was discharged once using a dispenser (Musashi, Japan) (refer to FIG. 21A). The discharged polymer composition was allowed to stand for drying for 2 minutes (refer to FIG. 21B) or 5 minutes (refer to FIG. 21C). A microstructure was formed on the pillar, and the microneedle-like microstructure was finally formed by a suitable extension method (e.g., drawing lithography).

According to an exemplary embodiment of the present invention, unlike conventional biodegradable microneedles with a pressure-sensitive adhesive patch, a biodegradable microstructure can be completely infiltrated into the skin, and the microstructure can be rapidly separated from the pillar part.

A candlelight-like microstructure can be formed by forming a pillar on a support and repeating contacting and drying, and may remain in the body by removing the infiltrated pillar.

The time required for leaving the microstructure in a body can be flexibly adjusted by changing a contact between the microstructure and the pillar or a dipping depth of the pillar.

Since a pillar has various lengths, when the structure for transdermal delivery is directly applied to a patient, a dermal infiltration depth can be flexibly adjusted.

Unlike conventional biodegradable microneedles having a pressure-sensitive adhesive patch, a microstructure can be infiltrated without interference by body hair and without shaving the infiltrated part.

While the present invention has been described with reference to the exemplary embodiments thereof, it should be understood that the idea of the present invention is not limited to the exemplary embodiments disclosed herein, and those of ordinary skill in the art who understand the idea of the present invention can easily suggest other exemplary embodiments by addition, alteration, deletion or modification of components without departing from the scope of the present invention.

What is claimed is:

1. A structure for transdermal delivery, which delivers a material into a body, comprising:
a pillar which is a first dermal infiltration part; and
a microstructure which is connected to an end of one side of the pillar and serves as a second dermal infiltration part,
wherein the microstructure comprises a body part formed of a biodegradable viscous composition; and
a connection part which includes a first contact surface having viscosity generated by a viscous composition to allow the body part to be connected to the pillar and disposed opposite the end of the one side of the pillar.

2. The structure of claim 1, wherein the connection part further comprises a second contact surface further providing a binding force between the microstructure and the pillar, the second contact surface comprising a part of the outer surface of the pillar extending from the first contact surface and having viscosity generated by the viscous composition.

3. The structure of claim 1, wherein the body part of the microstructure comprises a first body part extending from the first contact surface and a second body part connected with the first body part, in which the first body part is formed to have an increased cross-section from the first contact surface to the second body part, and the second body part is formed to have a decreased cross-section from the first body part to an end of the second body part.

4. The structure of claim 3, wherein the first body part and the second body part are formed in a substantially circular shape, and the first body part is formed to have a continuously increasing diameter from the first contact surface to the second body part.

5. The structure of claim 4, wherein the second body part is formed to have a decreased diameter from the first body part to an end of the second body part.

6. The structure of claim 5, wherein the second body part is formed to have a continuously decreasing diameter from the first body part to the end of the second body part.

7. The structure of claim 4, wherein, when a diameter of the pillar is D1 and a diameter of the connected surface of the first body part connected with the second body part is D2, the maximum diameter of the body part is D2, where D2>D1, and the diameter D of the first body part is continuously increased between D1 and D2.

8. The structure of claim 7, wherein, when a diameter of the first contact surface is D3, D1≦D3<D2.

9. The structure of claim 7, wherein the connected surface is placed in a substantially center of the microstructure in a lengthwise direction.

10. The structure of claim 3, wherein the second body part has a pointed end.

11. A method for manufacturing a structure for transdermal delivery having a microstructure formed of a viscous composition at an end of a projecting pillar and separated from the pillar within a predetermined separation time when applied to a body, the method comprising:
(a) providing a viscous composition containing a biocompatible or biodegradable material to be delivered into a body;
(b) providing a pillar with predetermined shape, length and diameter according to a characteristic of a finally-manufactured microstructure;
(c) determining a contact area in which the pillar is in contact with the viscous composition in consideration of the separation time for separating the viscous composition from the pillar in the body;
(d) placing the viscous composition at an end of the pillar by contacting the end of the pillar with the viscous composition by the determined contact area;
(e) drying the viscous composition placed at the end of the pillar; and
(f) forming the microstructure in which the viscous composition is attached to the end of the pillar by sequentially repeating the steps (c) to (e).

12. The method of claim 11, wherein the step (d) includes placing the viscous composition at the end of the pillar by discharging the viscous composition at at least a part of the end of the pillar.

13. The method of claim 11, wherein the step (d) includes placing the viscous composition at the end of the pillar and a part of the outer surface of the end of the pillar by dipping the end of the pillar in the viscous composition.

14. The method of claim 13, further comprising, between the step (d) and step (e), spacing the pillar dipped in the viscous composition from the viscous composition.

15. The method of claim 11, further comprising, after the step (f), applying an outward force to the viscous composition formed at the tip of the pillar before the viscous composition attached at the end of the pillar is solidified to reduce a diameter of the tip of the microstructure.

16. The method of claim 15, wherein the applying of the outward force is implemented by relatively moving the pillar in a vertical direction with respect to the support after the viscous composition formed at the tip of the pillar is brought into contact with the support.

17. The method of claim 15, wherein the applying of the outward force is implemented by treating the viscous composition by air blowing while the viscous composition formed at the tip of the pillar is in contact with a plate member and the pillar is relatively moved with respect to the plate member.

18. The method of claim 15, wherein the applying of the outward force is implemented by applying a centrifugal force to the viscous composition formed at the tip of the pillar.

19. The method of claim 15, wherein the applying of the outward force to the viscous composition is implemented by applying negative pressure to the viscous composition formed at the tip of the pillar.

20. The method of claim 11, wherein the step (f) is repeated sequentially from a viscous composition with a relatively small strength to a viscous composition with a relatively large strength.
